(19) United States
(12) Patent Application Publication
(10) Pub. No.: US 2022/0218767 A1
(43) Pub. Date: Jul. 14, 2022

(54) BLOOD GLUCOSE CONTROL AND ANTI-OBESITY PROBIOTICS COMPOSITIONS IN A SPECIFIC SELECTION AND RATIO

(71) Applicant: BE RICH BIOTECHNOLOGY CO., LTD., Taipei City (TW)

(72) Inventors: Cheng-Yuan Kao, Davis, CA (US); Chih-Cheng Tai, Los Gatos, CA (US); Amber Kao, Davis, CA (US)

(21) Appl. No.: 17/571,387

(22) Filed: Jan. 7, 2022

Related U.S. Application Data
(60) Provisional application No. 63/135,578, filed on Jan. 9, 2021.

(51) Int. Cl. A61K 35/747; A23L 33/135

(52) U.S. Cl. CPC A61K 35/747; A61K 2035/115; A23L 33/135

(57) ABSTRACT

One or more predetermined combination of human use (e.g., including animals) probiotics and specific ranges of ratio of anti-obesity human use probiotics compositions and their preparation methods and uses are provided. Particularly, the Present Disclosure provides a specific composition of food-grade probiotics been shaped by germ-free or antibiotics-treated dual-specificity phosphatase 6 (Dusp6) deficient mammal, which is effective in altering a relative abundance of gut microbiota and also useful in reducing body weight, fat mass, and/or size of adipocytes and increasing oxygen consumption and/or energy expenditure and thus can be used to treat or prevent obesity, manage blood glucose level, or their associated metabolic disorders or conditions in a subject in need.

### CROSS-REFERENCE TO RELATED APPLICATION(S)

The present invention claims priority to the U.S. provisional patent application Ser. No. 63/135,578, titled “ANTI-OBESITY PROBIOTICS COMPOSITIONS IN A SPECIFIC SELECTION AND RATIO,” filed on Jan. 9, 2021, which is incorporated by reference in its entirety for all purposes.

### FIELD OF INVENTION

The present invention relates to blood glucose control and anti-obesity human probiotics compositions. Particularly, the present invention relates to one or more preselected combinations and specific ranges of ratios of blood glucose control and anti-obesity human probiotics compositions and their preparation methods and uses.

### BACKGROUND OF THE INVENTION

Obesity is now a plague in developed countries as well as in many developing countries. Since obesity increases the risk of many health conditions, including cardiovascular disease, stroke, type 2 diabetes, fatty liver and certain cancers, it is important to understand the detailed mechanism of obesity development and search for novel ways to treat obesity.

Dual-specificity phosphatases (DUSPs) are canonically characterized as negative regulators of the mitogen-activated protein kinase (MAPK) pathway. Some studies have demonstrated that DUSP6, also known as MKP-3, negatively regulates ERK1/2 activity via dephosphorylation, although this dephosphorylation activity could be context-dependent. Dusp6-deficient mice have been shown to have enlarged hearts and an increased resistance to some heart diseases. It has been reported that Dusp6 is upregulated in the liver of obese and diabetic mice and promotes glucose output in both cultured liver cells and mouse livers. Furthermore, it has been shown that systemic Dusp6 deficiency could significantly decrease blood glucose levels, improve insulin sensitivity and increase the resistance to diet-induced obesity (DIO). Besides host genetic and environmental factors, the gut microbiota has been recognized as a major regulator in the development of obesity. However, whether or not dusp6-deficiency-mediated effects on obesity function via the gut microbiota remains unclear.

To decrease the risk from obesity and control the blood glucose, a method to treat or prevent obesity, or controlling the blood glucose is needed.

### SUMMARY OF THE INVENTION

For at least the above-mentioned purpose, one or more predetermined combinations of human (e.g., including animals) probiotics and specific ranges of ratio of anti-obesity human probiotics compositions and their preparation methods and uses are provided. Particularly, the present disclosure provides a specific composition of food-grade probiotics been shaped by germ-free or antibiotics-treated dual-specificity phosphatase 6 (Dusp6) deficient mammal, which is effective in altering a relative abundance of gut microbiota and also useful in reducing body weight, fat mass, and/or size of adipocytes and increasing oxygen consumption and/or energy expenditure and thus can be used to treat or prevent obesity or its associated metabolic disorders or conditions in a subject in need. The present disclosure also provides methods and compositions for stabilizing blood glucose levels, reducing blood glucose level spiking (e.g., within two hours of food consumption), and/or preventing blood glucose level fluctuation.

The present disclosure is to provide an anti-obesity/blood glucose control probiotic composition comprising:
a) two or more of probiotics selected from the group of:
   (i) substantially purified B. breve;
   (ii) substantially purified B. longum;
   (iii) substantially purified Streptococcus salivarius subsp. thermophilus;
   (iv) substantially purified L. acidophilus;
   (v) substantially purified L. casei; and
   (vi) substantially purified L. delbrueckii; and/or
b) carrier for hosting the two or more probiotics.

Preferably, the two or more of probiotics are in a predetermined percentage range ratio determined by a gut microbiota collected from a Dusp6 deficient mammal.

Preferably, the 6 food grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) are in a ratio of 1:1:1:1:1:1.

Preferably, the 6 food grade bacterial probiotics ((1) Bifidobacterium breve; (2) Bifidobacterium longum; (3) Streptococcus salivarius subsp. thermophiles; (4) Lactobacillus acidophilus; (5) Lactobacillus casei; and (6) Lactobacillus delbrueckii) are in a ratio of 10:10:10:1:1:1. A composition with a ratio of the above bacterial probiotics has a better and significant result than a result using an individual probiotic species above. In some embodiments, the ratio of the first three probiotics (1) Bifidobacterium breve, (2) Bifidobacterium longum, and (3) Streptococcus salivarius subsp. thermophiles is higher than the rest three probiotics (4) Lactobacillus acidophilus, (5) Lactobacillus casei, and (6) Lactobacillus delbrueckii. In some embodiments, the ratio of the first three probiotics (1) Bifidobacterium breve, (2) Bifidobacterium longum, and (3) Streptococcus salivarius subsp. thermophiles is at least 5 times in amount higher than the rest three probiotics (4) Lactobacillus acidophilus, (5) Lactobacillus casei, and (6) Lactobacillus delbrueckii. In other embodiments, the ratio of the first three probiotics (1) Bifidobacterium breve, (2) Bifidobacterium longum, and (3) Streptococcus salivarius subsp. thermophiles is at least 2 times in amount higher than the rest three probiotics (4) Lactobacillus acidophilus, (5) Lactobacillus casei, and (6) Lactobacillus delbrueckii. In some other embodiments, the ratio of the first three probiotics (1) Bifidobacterium breve, (2) Bifidobacterium longum, and (3) Streptococcus salivarius subsp. thermophiles is 2-100 times in amount higher than the rest three probiotics (4) Lactobacillus acidophilus, (5) Lactobacillus casei, and (6) Lactobacillus delbrueckii. In some embodiments, the ratio of the first three probiotics (1) Bifidobacterium breve, (2) Bifidobacterium longum, and (3) Streptococcus salivarius subsp. thermophiles is at least 1000 times in amount higher than the rest three probiotics (4) Lactobacillus acidophilus, (5) Lactobacillus casei, and (6) Lactobacillus delbrueckii.

Preferably, the probiotics is a mixture of (i) substantially purified B. breve and (ii) substantially purified B. longum, wherein the percentage of (i) substantially purified B. breve is from 30% to 70%, and the percentage of (ii) substantially purified B. longum is from 70% to 30%.

Preferably, the probiotic is a mixture of (i) substantially purified B. breve, (ii) substantially purified B. longum and (vi) substantially purified L. delbrueckii, wherein the percentage of (i) substantially purified B. breve is from 30% to 40%, the percentage of (ii) substantially purified B. longum is from 30% to 40%, and the percentage of (vi) substantially purified L. delbrueckii is from 20% to 40%.

Preferably, the carrier comprises nutrition, adjuvant, and/or filler, such as yogurt, soy-based products, cereal-based products, fruits, vegetables and meat products. A person of ordinary skilled in the art appreciates any dairy and nondairy-based products contain suitable amounts of carbohydrates, fibers, proteins and vitamins that support the growth of probiotics are within the scope of the present disclosure.

Preferably, an application form of the composition comprises powder, liquid, gel, tablet, pills, and slurry.

Preferably, the composition is formulated as a food product, dietary supplement or medicament. In some aspect, the present disclosure provides a method of reducing body weight and/or body fat and/or preventing an increase in body weight and/or body fat in a subject thereof by administering to the subject an effective amount of the composition as descried herein. The present disclosure also provides methods and compositions for stabilizing blood glucose levels, reducing blood glucose level spiking (e.g., within two hours of food consumption), and/or preventing blood glucose level fluctuation.

Still another purpose of the present disclosure is to provide a method for treating or preventing obesity or its associated disorders or conditions in a subject thereof by administering to the subject an effective amount of the composition as described herein.

Preferably, the obesity to be treated is diet-induced obesity (DIO).

Preferably, the obesity associated disorders or conditions include type 2 diabetes, hyperglycemia, glucose intolerance, dyslipidemia, insulin resistance, hyperinsulinemia, fatty liver, cardiovascular disease, stroke, and cancer. Thus, the present disclosure provides the selected compositions and/or ratio of probiotics in treating or preventing the obesity associated disorders and conditions.

Another purpose of the present disclosure is to provide a food product, which comprises the anti-obesity probiotic composition.

Preferably, the food product is yogurt, jelly, cream or cheese.

Preferably, the food product further comprising food additives, such as vitamin, calcium or cellulose.

One purpose of the present disclosure is a method of forming a probiotic composition comprising:
a) preparing a carrier;
b) selecting two or more probiotics at a predetermined ratio, wherein the two or more probiotics comprises (i) Bifidobacterium breve; ii) Bifidobacterium longum; iii) Streptococcus salivarius subsp. thermophiles; iv) Lactobacillus acidophilus; v) Lactobacillus casei; and vi) Lactobacillus delbrueckii;
c) mixing the at least two or more probiotics with the carrier; and
d) forming the probiotic composition by coupling the at least two or more probiotics with the carrier.

Preferably, the method further comprising encapsuling the probiotic composition in a capsule or tablet.

Preferably, the method further comprising mixing the at least two or more probiotics with one or more prebiotics.

Preferably, the prebiotics comprises CAMU CAMU and betaine.

Another purpose of the present disclosure is a method of stabilizing blood glucose level comprising:
a) taking food;
b) controlling or alleviating a symptom by taking an effective dosage of a probiotic composition having two or more of the probiotics at a predetermined ratio, wherein the two or more probiotics comprises (i) Bifidobacterium breve; ii) Bifidobacterium longum; iii) Streptococcus salivarius subsp. thermophiles; iv) Lactobacillus acidophilus; v) Lactobacillus casei; and vi) Lactobacillus delbrueckii.

Preferably, the method further comprising reducing or preventing an increase a body weight of a human or animal by taking the effective dosage daily.

Preferably, the symptom comprises obesity associated disorders.

Preferably, the symptom comprises conditions include type 2 diabetes, hyperglycemia, glucose intolerance, dyslipidemia, insulin resistance, hyperinsulinemia, fatty liver, cardiovascular disease, stroke, and cancer.

Preferably, the method further comprising preventing spiking of a blood glucose level by taking the effective dosage after food consumption.

Preferably, the method further comprising stabilizing a blood glucose level by taking the effective dosage in a routine interval.

Preferably, the method further comprising forming a food product in a form of yogurt, jelly, cream or cheese.

Preferably, the method further comprising mixing vitamin, calcium or cellulose.

With the above-mentioned technical features, a composition having a predetermined percentage range ratio determined by a gut microbiota collected from, selected through, shaped by, or established using a Dusp6 deficient mammal, and method or treatment for decreasing the risk of obesity or controlling the blood glucose may be provided.

### BRIEF DESCRIPTION OF THE DRAWINGS

FIG. 1 illustrates a probiotic microbiota obtaining method 100 in accordance with an embodiment of the present disclosure.

FIG. 2 illustrates a diagram showing the time progress of the probiotic microbiota obtaining method in accordance with an embodiment of the present disclosure.

FIG. 3 illustrates the result of change of Lee index, BMI and organ weight of the wild type mice and D6KO mice before and after the method 100 in FIG. 1.

FIG. 4 illustrates the abundance of the intestinal bacteria of wild type and D6KO mice at T0 and T10.

FIG. 5 illustrates the Non-metric Multidimensional Scaling of the intestinal bacteria of wild type and D6KO mice at T0 and T10.

FIG. 6 illustrates a diagram of the IP-Glucose Tolerance Test (GTT)(mg/dL).

### DETAILED DESCRIPTION OF THE INVENTION

The present disclosure is based on the unexpected results that gut microbiota collected from a dual-specificity phosphatase 6 (Dusp6) deficient mammal, when administrated into a subject, can change relevant abundance of microbiota in a gastrointestinal tract of the subject. The present disclosure also provides that the gut microbiota collected from a Dusp6 deficient mammal exhibit anti-obesity activities under high-fat diet, which are effective in reducing of body weight, fat mass, and/or size of adipocytes and increasing oxygen consumption and/or energy expenditure, and thus can be used to treat obesity or its associated disorders or conditions in a subject in need. The present disclosure also provides a platform technology to obtain a combination of 6 food grade bacterial probiotics with specific ratio by using a Dusp6 deficient mammal therefrom for treatment/prevention of obesity or its associated disorders or conditions.

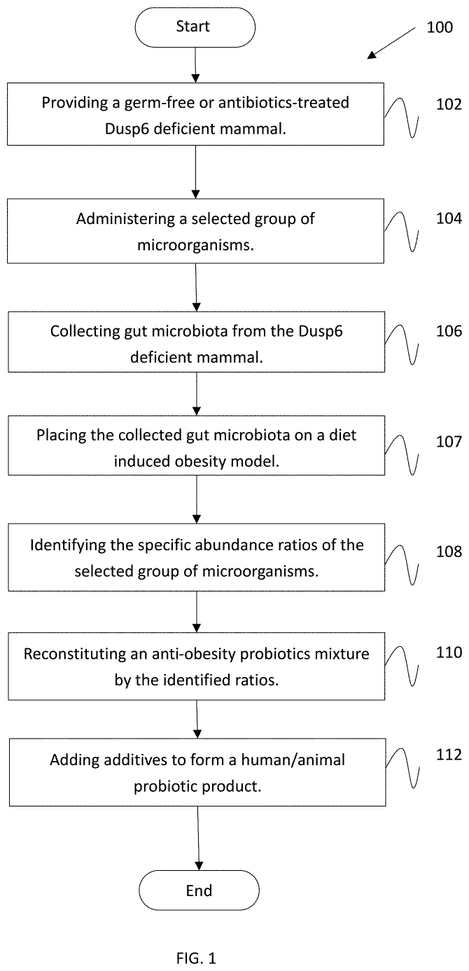

FIG. 1 illustrates a probiotic microbiota obtaining method 100 in accordance with some embodiments. At a Step 102, a germ-free or antibiotics-treated Dusp6 deficient mammal is provided. At a Step 104, a selected group of microorganisms are administrated to the germ-free or antibiotics-treated Dusp6 deficient mammal. In one embodiment, 6 food grade bacterial probiotics (in equal amounts of colony-forming units) are orally administrated into a germ-free Dusp6 deficient mammal. The 6 food grade bacterial probiotics include 1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii.

At a Step 106, gut microbiota from the Dusp6 deficient mammal are collected.

At a Step 107, the collected gut microbiota are placed on a diet induced obesity model.

At a Step 108, specific abundance ratios of these 6 food-grade bacterial probiotics mixture shaped by Dusp6 deficient mammal is identified. At a Step 110, an anti-obesity/glucose stabilizing probiotics mixture by the identified ratios is reconstituted. At a Step 112, additives are added to form a human/animal probiotic product.

Experimental Section for Mice Treatment

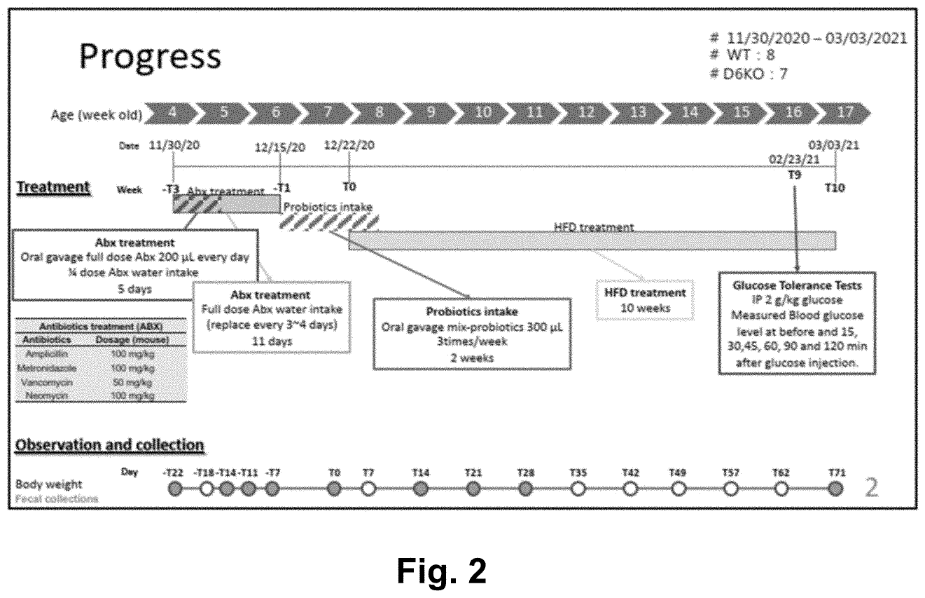

FIG. 2 illustrates a diagram showing the time progress of the probiotic microbiota obtaining method in accordance with embodiments of the present disclosure.

In some embodiments, groups of 4 weeks old 8 wild type mice and 7 Dusp6-deficient mice (D6KO) are used. In some embodiments, the full dose of Abx is comprised with antibiotics water (ampicillin 100 mg/kg of mouse weight, metronidazole 100 mg/kg of mouse weight, vancomycin 50 mg/kg of mouse weight, neomycin 100 mg/kg of mouse weight) for native gut microbiota depletion. The mice are initially treated with first stage Abx (antibiotics treatment), which comprises oral gavage full dose Abx 200 uL every day and ¼ dose Abx in drinking water for 5 days (begins at −T3). Then, the second stage Abx treatment changes to full dose Abx water intake (replaced every 3˜4 days) for 11 days (until −T1). The Abx treatment removes most of the microbiota in a gastrointestinal tract to obtain gut-microbiota-depleted mice. The Abx treatment here is merely one of choices to remove native microbiota in a gastrointestinal tract, the disclosure is not limited thereof. Any possible method to create gut-microbiota-depleted mice is able to be used.

Probiotics Mixture Preparation

The mixture of the probiotics are able to be prepared having the amount as show in table 1.

TABLE 1

| | | Resuspended Dosage |
|---|---|---|
| | Manufactured | (6.0 × 10e9/mouse/ |
| | Dosage | probiotics/300 uL PBS) |
| Bifidobacterium breve | ≥1.0 × 10¹¹ CFU/g | 0.2 g/1000 uL PBS |
| (e.g., BR18) | | |
| Bifidobacterium longum | ≥1.0 × 10¹¹ CFU/g | 0.2 g/1000 uL PBS |
| (e.g., BL986) | | |
| Lactobacillus acidophilus | ≥1.0 × 10¹¹ CFU/g | 0.2 g/1000 uL PBS |
| (e.g., LA1063) | | |
| Lactobacillus casei | ≥1.0 × 10¹¹ CFU/g | 0.2 g/1000 uL PBS |
| (e.g., LC122) | | |
| Lactobacillus delbrueckii | ≥1.0 × 10¹¹ CFU/g | 0.2 g/1000 uL PBS |
| subsp. lactis (e.g., LDL114) | | |
| Streptococcus | ≥1.0 × 10¹¹ CFU/g | 0.2 g/1000 uL PBS |
| thermophilus (e.g., ST37) | | |

The probiotics intake begins at −T1, which comprises oral gavage mix-probiotics 300 uL 3 times/week for 2 weeks. Then all mice are treated with probiotics mix including Bifidobacterium breve (e.g., Bifidobacterium breve BR18), Bifidobacterium longum (e.g., Bifidobacterium longum BL986), Lactobacillus acidophilus (e.g., Lactobacillus acidophilus LA1063), Lactobacillus casei (e.g., Lactobacillus casei LC122), Lactobacillus delbrueckii subsp. lactis (e.g., Lactobacillus delbrueckii subsp. lactis LDL114) and Streptococcus thermophilus (e.g., Streptococcus thermophilus ST37) in only one dosage of equal ratio, the 45% calories fat HFD treatment (high-fat diet treatment) begins at T0 for 10 weeks. The HFD treatment is used to perform diet-induced obesity.

Observation and Collection

The body weights of the mice are observed at day −22, −18, −14, −11, −7, and about once/week from day 0 to day 71. Also, the fecal collections are performed at day −22, −14, −11, −7, 0, 14, 21, 28 and 71.

Glucose tolerance test is also performed at T9, which comprises intraperitoneal injection 2 g/kg glucose to mice, and measure blood glucose level at before and 15, 30, 45, 60, 90, and 120 min after glucose injection.

Results

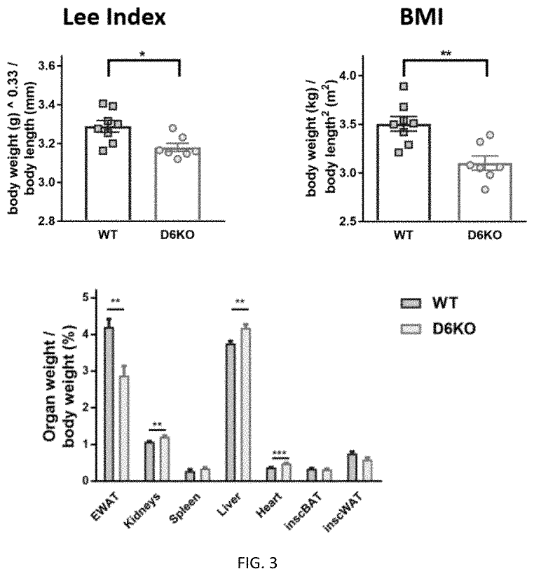

See FIG. 3. FIG. 3 illustrates the result of change of Lee index, BMI and organ weight of the wild type mice and D6KO mice before and after the method 100 of FIG. 1.

The estimation of body fat may be calculated by Lee index and BMI of the mice.

Both Lee index and BMI of the D6KO mice are statistically significantly lower than the wild-type mice after a 10 weeks high-fat-diet treatment. The result is further supported by our experiments showing that the microbiota in D6KO is able to attenuate the high-fat-diet-induced weight gain in wildtype mice (Nat Microbiol. 2016 Nov. 28; 2:16220. doi: 10.1038/nmicrobiol.2016.220.), which is incorporated by reference for all purposes.

Organ Weights

The epididymal white adipose tissue (EWAT) of the D6KO mice are statistically significantly lower in weight than the wild-type mice after a 10 weeks high-fat-diet treatment.

Diversity and Abundance of Intestinal Microbiota

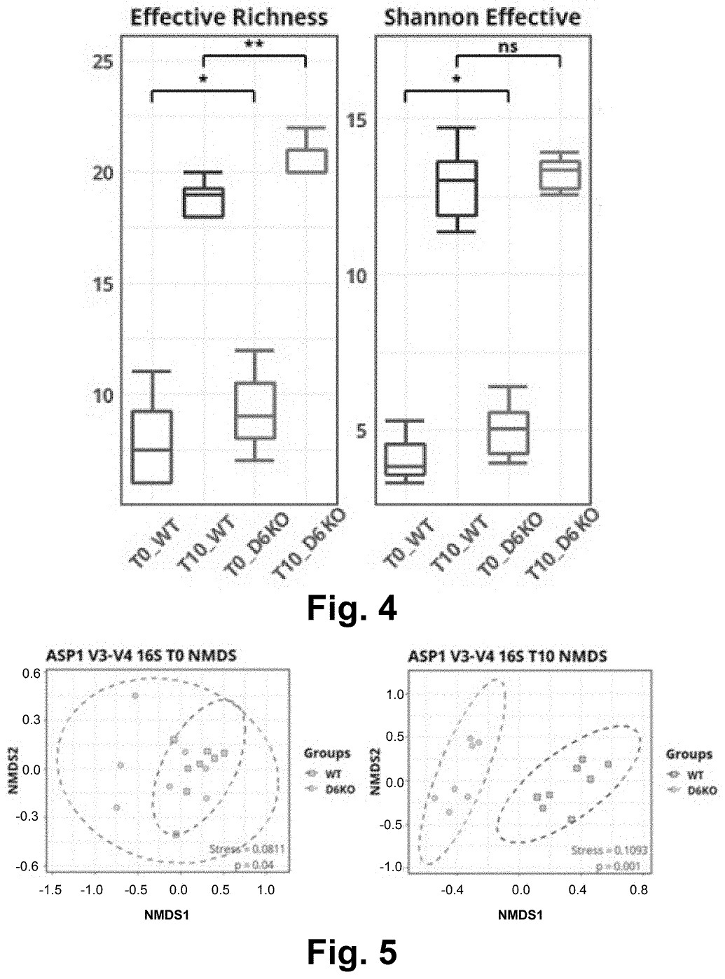

See FIGS. 4 and 5, FIG. 4 illustrates the abundance of the intestinal bacteria of wild type and D6KO mice at T0 and T10. FIG. 5 illustrates the Non-metric Multidimensional Scaling of the intestinal bacteria of wild type and D6KO mice at T0 and T10.

Effective richness, Shannon index and beta-diversity NMDS are calculated by Rhea (V1.6) R pipeline.

The data of the intestinal bacteria of wild type and D6KO mice at T0 and T10 performs NMDS, using Bray-Curtis distance for calculation and the number of verification replacements is 999.

As the result, the richness and alpha-diversity of D6KO mice gut microbiome is greater than wild type mice. A moderate degree to profound gut microbiome dysbiosis is associated with obesity and metabolic disorders such as type-2 diabetes. The increase of gut microbiome richness and alpha-diversity is in general linked to an overall better health condition.

The Dusp6-deficient mice are resistant to diet-induced obesity. The fecal microbiota transplant experiments in germ-free mouse models showed that the gut microbiota contribute substantially to the diet-induced-obesity-resistant phenotype in Dusp6 knockout mice.

Abundance of the Six Probiotics Mixture Treatments

Based on the results described above, D6KO mice are still more resistant to diet-induced obesity even their native gut microbiota is replaced to 6-probiotics mix. It is shown that D6KO mice and wild type mice with the 6-probiotics mix are more resistant to diet-induced obesity and have better results in blood glucose control.

The relative ratios of the 6 food grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) in oral gavage to wild-type mic and D6KO mice are in a ratio of 1:1:1:1:1:1.

Regarding to the relative ratios of the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) in D6KO mice before HFD (T0), are in a ratio of 56:8712:836:46:350:0 based on the qPCR analysis. In some embodiments, the ratio of the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii are in the range of 1:100-200:10-20:0.1-1:1-10:0-10, which indicates significant effects in weight reduction, blood glucose control/stabilization among other positive results.

Regarding to the relative ratios of the 6 food grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) in D6KO mice before HFD (T0), are in a ratio of 56:8713:836:46:350:0 based on the qPCR analysis.

Regarding to the relative ratios of the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) in D6KO mice after HFD (T10), are in a ratio of 1:1055:20:0:0:8921 based on the qPCR analysis. In some embodiments, the ratio of the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii are in the range of 1:100-200:10-20:0.1-1:1-10:0-10, which indicates significant effects in weight reduction, blood glucose control/stabilization among other positive results.

Intraperitoneal Glucose Tolerance Test (IP-GTT)

In another embodiment, 8 mice are prepared for each of the control group and the treatment group. The 8 mice are germ-free mice. Gavage of the control group mice is provided with ddH₂O (300 uL/time, 3 times/week) and fed with HFD, and gavage of the treatment mice is provided with 1% Betaine+200 mg/kg CAMU CAMU+1.0×109 CFU/per probiotics of the 6 food grade probiotics, and fed with HFD.

The IP-GTT test is performed at 6^{th} week of high-fat-det treatment. The process of the IP-GTT test is as follow. Mice were fasted for 12 hours with free access to water, and intraperitoneally injected with glucose at a dose of 2 g per kg body weight. Blood glucose level was measured with a glucometer (Johnson & Johnson) immediately before and 15, 30, 45, 60, 90 and 120 min after glucose injection. In the GTT response plot, the y-axis is the blood glucose concentration and the x-axis is the time that the blood glucose was measured. In the AUC plot, the y-axis means the The Area Under the Curve (AUC) derived from the IP-GTT by calculating the incremental Area Under Curve (AUC) is developed is used to compare the impaired glucose tolerance levels in different groups of mice. The Y-axis is the arbitrary unit of Area Under Curve and the X-axis is the different groups of mice.

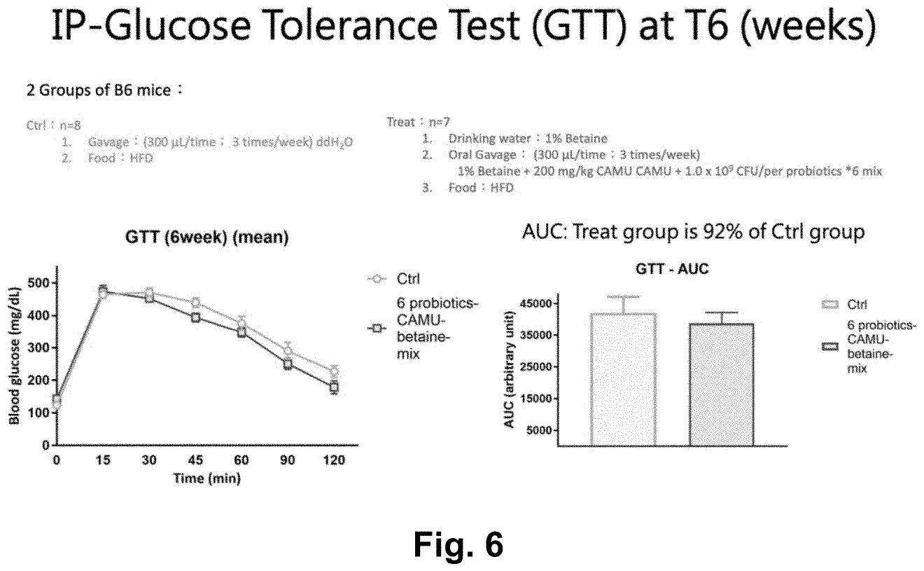

See FIG. 6, FIG. 6 illustrates a diagram of the IP-Glucose Tolerance Test (GTT)(mg/dL) at 6^{th} week.

As shown in FIG. 6, the glucose tolerance of the treated mice is 8% better than the control group. The result shows that the mice treated with 1% Betaine+200 mg/kg CAMU CAMU+1.0×109 CFU/per probiotics of the 6 food grade probiotics may have higher tolerance to the change of blood glucose level.

Since the probiotics may help controlling the blood glucose level, it may be administered at a) after detecting a spike in blood sugar, b) daily life (e.g., as daily use supplement), or to c) pregnant women and other people who cannot use insulin.

In some embodiments, the probiotics may be applied with effective dose about 1-100 billion CFU/day. Preferably, the probiotics may be applied with 10-50 billion CFU/day. More preferably, the probiotics may be applied with 30 billion CFU/day.

In some embodiments, the 6 food grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) are in a ratio of 1:1:1:1:1:1.

In a high-fat-diet induced obesity mouse model with wild-type mice, the administration of 6-probiotics mix ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii in a 1:1:1:1:1:1:1 ratio) with Camu Camu and Betaine for 6-weeks enhances ˜8% of the glucose homeostasis based on glucose tolerance test.

In contrast, it is reported that a typical combination or individual species of Lactobacillus acidophilus and Bifidobacterium longum alone do not show any effect on glucose homeostasis (Biochem Biophys Res Commun 2016 Nov. 11; 480(2):222-227. doi: 10.1016/j.bbrc.2016.10.031.).

Similarly, typical Lactobacillus delbrueckii administration by itself in a genetic type-2-diabetic mouse model has shown no effect on glucose homeostasis (J Clin Biochem Nutr. 2012 September; 51(2):96-101. doi: 10.3164/jcbn.11-07.).

In some embodiments, the combination of the 6 food-grade bacterial probiotics has at least selected 1, 2, 3, 4, or 5 of the 6 probiotics that is 10 times, 100 times or 1000 times more than the rest of probiotics.

In some embodiments, the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) are in a ratio of 10:10:1:10:10:1.

Method for Determining the Ratio of the Probiotics is qPCR Analysis Using General 16S rRNA Sequence as a Reference to Calculate the Relative Abundance.

In some embodiments, the method further comprises measuring the 6 food-grade bacterial probiotics mixture's anti-obesity activity in a subject.

In some embodiments, the 6 food-grade bacterial probiotics mixture are identified by measuring whether the microbes are effective in reducing body weight, fat mass, inducing ucp-1 browning gene expression and/or size of adipocytes in a subject upon administration of the microbes.

In some embodiments, the 6 food-grade bacterial probiotics mixture are identified by measuring whether the microbes are effective in increasing oxygen consumption and/or energy expenditure in a subject upon administration of the microbes.

Composition Including the Probiotics

Accordingly, the present invention also provides a composition, which comprises (i) substantially purified B. breve, (ii) substantially purified B. longum, (iii) substantially purified Streptococcus salivarius subsp. thermophilus, (iv) substantially purified L. acidophilus, (v) substantially purified L. casei, and (vi) substantially purified L. delbrueckii.

In some embodiments, the composition is obtained from a Dusp6 deficient mammal according to a method as described herein.

In some embodiments, the composition is formulated as a food product, dietary supplement or medicament.

In some embodiments, the composition is formulated as powder, liquid, gel, tablet, pills, and any other forms for human use.

In some embodiments, the composition is for use in altering a relative abundance of microbiota in a subject.

Treatment to Prevent from Obesity or its Associated Disorders or Conditions

In some embodiments, the composition is for use in reducing body weight and/or body fat, preventing an increase in body weight and/or body fat, and/or treating or preventing obesity or its associated disorders or conditions in a subject.

In some embodiments, a method is provided for using a composition as described herein. Such as a selection of any numbers (such as, 1, 2, 3, 4, 5, or 6) of the probiotics from the 6 exemplary probiotics disclosed herein to be used as selected or to be mixed with any other probiotics, prebiotics, or any other nutrients or substances, including CAMU CAMU and betaine A person of ordinary skilled in the art will appreciate any other substances that can enhance the effects disclosed here are within the scope of the Present Disclosure, such as other probiotics and prebiotics.

In particular, the present disclosure provides a method of altering a relative abundance of microbiota in a subject in need thereof by administering to the subject an effective amount of a composition as described herein. In some embodiments, the present disclosure provides a composition as described herein for manufacturing a food product, dietary supplement or medicament for altering a relative abundance of microbiota in a subject in need thereof.

The present disclosure also provides a method of reducing body weight and/or body fat and/or preventing an increase in body weight and/or body fat in a subject thereof by administering to the subject an effective amount of a composition as described herein.

The present disclosure also further provides a method for treating or preventing obesity or its associated disorders or conditions in a subject thereof by administering to the subject an effective amount of a composition as described herein. The present disclosure provides a composition as described herein for manufacturing a food product, dietary supplement or medicament for reducing body weight and/or body fat, preventing an increase in body weight, and/or body fat and/or treating or preventing obesity or its associated disorders or conditions in a subject in need thereof.

In some embodiments, the obesity to be treated is diet induced obesity (DIO).

In some embodiments, the obesity associated disorders or conditions include type 2 diabetes, hyperglycemia, glucose intolerance, dyslipidemia, insulin resistance, hyperinsulinemia, fatty liver, cardiovascular disease, stroke, and cancer. Thus, the present disclosure provides the selected compositions and/or ratio of probiotics in treating or preventing the obesity associated disorders and conditions.

In one embodiment, the food or drink product may be yogurt. Yogurt is good for health and may be easily purchased in daily life. The yogurt may comprise the 6 food grade bacterial probiotics mixture. In other embodiments, the yogurt may comprise at least 2 of the 6 food grade bacterial probiotics mixture, such as only comprising the mixture of Bifidobacterium breve (e.g., Bifidobacterium breve BR18) and Bifidobacterium longum (e.g., Bifidobacterium longum BL986), or comprising Bifidobacterium breve (e.g., Bifidobacterium breve BR18), Bifidobacterium longum (e.g., Bifidobacterium longum BL986) and Lactobacillus delbrueckii subsp. lactis (e.g., Lactobacillus delbrueckii subsp. lactis LDL114). The present disclosure is not limited to it, any possible composition consisting the 6 food grade bacterial probiotics may be used. The yogurt may further comprise vitamin, calcium, cellulose or other natural product as food additives as needed.

Since the probiotics are add into yogurt, the mixture may be intake more easily in daily life. In other embodiment, the mixture may be provided in cheese, jelly, cream or any suitable food.

In operation, determining specific probiotics group or specific ratios; using the selected probiotics group or specific ratios to make a dietary supplement or medication; and using the selected probiotics as recommended.

To decrease the risk from obesity, the present disclosure further provides a food or drink product comprising the above mentioned probiotics.

In utilization, the methods and compositions are able to be used to control and/or stabilize blood glucose level and reduce gaining weight and fat.

The description is presented to enable one of ordinary skill in the art to make and use the invention. Various modifications to the described embodiments are readily apparent to those persons skilled in the art and the generic principles herein can be applied to other embodiments. Thus, the present invention is not intended to be limited to the embodiments shown but is to be accorded the widest scope consistent with the principles and features described herein. It is readily apparent to one skilled in the art that other modifications can be made to the embodiments without departing from the spirit and scope of the invention as defined by the appended claims.

What is claimed is:

1. A probiotic composition comprising:
a) two or more of probiotics selected from the group of:
(i) substantially purified B. breve;
(ii) substantially purified B. longum;
(iii) substantially purified Streptococcus salivarius subsp. thermophilus;
(iv) substantially purified L. acidophilus;
(v) substantially purified L. casei; and
(vi) substantially purified L. delbrueckii; and
b) carrier for hosting the two or more probiotics.

2. The probiotic composition of claim 1, wherein the two or more of probiotics are in a predetermined percentage range ratio determined by a gut microbiota collected from a Dusp6 deficient mammal.

3. The probiotic composition of claim 1, wherein the two or more of probiotics are processed from 6 food-grade bacterial probiotics that are ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii).

4. The probiotic composition of claim 3, wherein the 6 food-grade bacterial probiotics are in a ratio of 1:1:1:1:1:1.

5. The probiotic composition of claim 3, wherein the 6 food-grade bacterial probiotics ((1) Bifidobacterium breve; 2) Bifidobacterium longum; 3) Streptococcus salivarius subsp. thermophiles; 4) Lactobacillus acidophilus; 5) Lactobacillus casei; and 6) Lactobacillus delbrueckii) are in a ratio of 10:10:10:1:1:1.

6. The probiotic composition of claim 1, wherein the probiotics is a mixture of of (i) Bifidobacterium breve and (ii) Bifidobacterium longum, wherein the ratio of (i) Bifidobacterium breve and (ii) Bifidobacterium longum is 7:3.

7. The probiotic composition of claim 1, wherein the probiotics is a mixture of (i) Bifidobacterium breve, (ii) Bifidobacterium longum and (iii) Streptococcus salivarius subsp. thermophiles, wherein the ratio of (i) Bifidobacterium breve, (ii) Bifidobacterium longum and (iii) Streptococcus salivarius subsp. thermophiles is 3:3:4.

8. The probiotic composition of claim 1, wherein the carrier comprises: a nutrient, an adjuvant, or a filler.

9. The probiotic composition of claim 1, wherein an application form of the composition comprises powder, liquid, gel, tablet, pills.

10. The probiotic composition of claim 1, wherein the composition is formulated as a food product, dietary supplement or medicament.

11. A probiotic composition comprising:
(a) carrier particles coupling with at least two of probiotics; and
(b) the at least two probiotics are selected from a group of:
i) Bifidobacterium breve;
ii) Bifidobacterium longum;
iii) Streptococcus salivarius subsp. thermophiles;
iv) Lactobacillus acidophilus;
v) Lactobacillus casei; and
vi) Lactobacillus delbrueckii.

12. The probiotic composition of claim 11, wherein the at least two of probiotics are substantially purified probiotics.

13. The probiotic composition of claim 11, wherein the at least two of probiotics are in a weight ratio of 1:1.

14. The probiotic composition of claim 11, wherein the at least two of probiotics are in a weight ratio of 10:1.

15. The probiotic composition of claim 11, wherein the at least two of probiotics are in a weight ratio of 0-30% and 31%-100%.

16. A method of forming a probiotic composition comprising:
a) preparing a carrier;
b) selecting two or more probiotics at a predetermined ratio, wherein the two or more probiotics comprises
i) Bifidobacterium breve;
ii) Bifidobacterium longum;
iii) Streptococcus salivarius subsp. thermophiles;
iv) Lactobacillus acidophilus;
v) Lactobacillus casei; and
vi) Lactobacillus delbrueckii;
b) mixing the at least two or more probiotics with the carrier; and
c) forming the probiotic composition by coupling the at least two or more probiotics with the carrier.

17. The method of claim 16, further comprising encapsuling the probiotic composition in a capsule or tablet.

18. The method of claim 16, further comprising mixing the at least two or more probiotics with one or more prebiotics.

19. The method of claim 18, where in the prebiotics comprises CAMU CAMU and betaine.

20. A method of stabilizing blood glucose level comprising:
a) taking food;
b) controlling or alleviating a symptom by taking an effective dosage of a probiotic composition having two or more of the probiotics at a predetermined ratio, wherein the two or more probiotics comprises (i) Bifidobacterium breve; ii) Bifidobacterium longum; iii) Streptococcus salivarius subsp. thermophiles; iv) Lactobacillus acidophilus; v) Lactobacillus casei; and vi) Lactobacillus delbrueckii.

21. The method of claim 20, further comprising reducing or preventing an increase a body weight of a human or animal by taking the effective dosage daily.

22. The method of claim 20, wherein the symptom comprises obesity associated disorders.

23. The method of claim 20, wherein the symptom comprises conditions include type 2 diabetes, hyperglycemia, glucose intolerance, dyslipidemia, insulin resistance, hyperinsulinemia, fatty liver, cardiovascular disease, stroke, and cancer.

24. The method of claim 20, further comprising preventing spiking of a blood glucose level by taking the effective dosage after food consumption.

25. The method of claim 20, further comprising stabilizing a blood glucose level by taking the effective dosage in a routine interval.

26. The method of claim 20, further comprising forming a food product in a form of yogurt, jelly, cream or cheese.

27. The method of claim 20, further comprising mixing vitamin, calcium or cellulose.
